(19) United States
(12) Patent Application Publication
(10) Pub. No.: US 2022/0204920 A1
(43) Pub. Date: Jun. 30, 2022

(54) METHOD FOR REDUCING METHIONINE OXIDATION IN RECOMBINANT PROTEINS

(71) Applicant: FORMYCON AG, Martinsried/Planegg (DE)

(72) Inventor: Christian ECKERT, Andechs (DE)

(21) Appl. No.: 17/610,894

(22) PCT Filed: May 14, 2020
(86) PCT No.: PCT/EP2020/063424

§ 371 Date: Nov. 12, 2021

(30) Foreign Application Priority Data
May 16, 2019 (EP) 19174976.1

(51) Int. Cl. C12N 5/00

(52) U.S. Cl. CPC C12N 5/0031; C12N 2511/00; C12N 2500/32

(57) ABSTRACT

The present invention relates to a method for reducing the oxidation of methionine residues in a recombinant protein by culturing cells expressing the recombinant protein in a cell culture medium and feeding the cells with glutamine.

### FIELD OF THE INVENTION

The present invention relates to a method for reducing the oxidation of methionine residues in a recombinant protein by culturing cells expressing the recombinant protein in a cell culture medium and feeding the cells with glutamine.

### BACKGROUND OF THE INVENTION

Proteins may undergo varying degrees of degradation during their manufacture. One of the major degradation pathways of proteins is oxidation which has a negative effect on protein stability and potency. Oxidative reactions may cause destruction of amino acid residues, peptide bond hydrolysis, and hence protein instability due to alteration of the protein's tertiary structure and protein aggregation (Davies (1987) J. Biol. Chem. 262: 9895-901). The oxidation of protein pharmaceuticals has been reviewed by Nguyen (Chapter 4 in Formulation and Delivery of Protein and Peptides (1994)), Hovorka (2001) J. Pharm Sci. 90:25369 and Li (1995) Biotech Bioengineering 48:490-500.

Oxidation of proteins such as monoclonal antibody-containing solutions can result in degradation, aggregation and fragmentation of the antibody, and thus loss of antibody activity. In other cases, even though the protein pharmaceutical is still biologically active after oxidation, the protein may not be acceptable for pharmaceutical use according to the standards of regulatory agencies, for example, if high levels of methionine sulfoxide are present.

The side chains of the amino acids cysteine (Cys), methionine (Met), tryptophan (Trp), histidine (His), and tyrosine (Tyr) residues are particularly prone to oxidation. The oxidation of methionine residues leads to the substitution of the thioether group with a sulfoxide group. It has been shown that the pharmaceutical proteins human growth hormone, IL-2, relaxin, recombinant human GCSF, recombinant human VEGF, IGF-I, recombinant human CNF, recombinant human NGF, BDNF, recombinant human leptin, herceptin and PTH exhibit methionine oxidation.

WO 2007/003640 A1 describes the production of recombinant follicle-stimulating hormone (FSH) in a serum-free culture medium comprising an antioxidant selected from L-glutathione, 2-mercaptoethanol, L-methionine and a combination of ascorbic acid and (+)-alpha-tocopherol which leads to a reduction of oxidized forms of FSH.

Nevertheless, there is still a need to identify culture conditions which reduce the formation of oxidized methionine residues during the production of a recombinant protein in host cells.

### SUMMARY OF THE INVENTION

The present inventor have surprisingly found that the feeding of cells expressing a recombinant protein with glutamine significantly reduces the percentage of oxidized methionine residues in the recombinant protein.

Hence, the present invention relates to a method for reducing the percentage of oxidized methionine residues in a recombinant protein, said method comprising culturing cells expressing said recombinant protein in a suitable cell culture medium and feeding the cells with glutamine.

In one embodiment the method comprises feeding the cells with 0.2 mM to 5 mM glutamine.

In one embodiment the method comprises feeding the cells with 0.5 mM to 4 mM glutamine on day 3 to 5 of the culture.

In one embodiment the method comprises feeding the cells with 0.2 mM to 2 mM glutamine on day 6 to 12 of the culture.

The method may further comprise feeding the cells with methionine.

In one embodiment the method comprises feeding the cells with 250 to 1000 mg/l methionine.

The method may further comprise reducing the culture temperature.

The culture temperature may be reduced from 37° C. to a temperature within the range of 30° C. to 35° C.

The culture temperature may be reduced in a first step from a first temperature of 37° C. to a second temperature within the range of 33° C. to 35° C. and in a second step from the second temperature of 33° C. to 35° C. to a third temperature within the range of 31° C. to 33° C.

The recombinant protein may be a recombinant antibody or a fusion protein of a receptor and the Fc portion of an immunoglobulin and in one embodiment it is aflibercept.

The cells may be CHO cells.

The present invention also relates to the use of glutamine for reducing the percentage of oxidized methionine residues in a composition comprising a recombinant protein.

The present invention also relates to a method for producing a recombinant protein, comprising
(a) culturing cells expressing the recombinant protein in a suitable cell culture medium;
(b) feeding the cells with glutamine, wherein the cells are fed with 0.5 mM to 4 mM glutamine on day 3 to 5 of the culture and with 0.2 mM to 2 mM glutamine on day 6 to 12 of the culture; and
(c) obtaining the recombinant protein.

The present invention also relates to a method for producing aflibercept, comprising:
(a) culturing cells expressing aflibercept in a suitable cell culture medium;
(b) feeding the cells with glutamine; and
(c) obtaining the aflibercept.

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As discussed above, the present invention is based on the finding that feeding cells expressing a recombinant protein with glutamine reduces the oxidation of methionine residues within the recombinant protein.

The terms “oxidized methionine residues” and “oxidation of methionine residues” mean that the thioether group within a methionine residue in the recombinant protein is oxidized so that methionine sulfoxide is formed. If the recombinant protein is the VEGFR-Fc fusion protein aflibercept, the methionine residue which is oxidized may be methionine 192 which is located in the VEGF receptor 2 part of the protein. If the recombinant protein is a full-length IgG1 antibody, the methionine residues which are oxidized may be Met 256 and Met432 which are located in the antibody CH2-CH3 interface. If the recombinant protein is human growth hormone, the methionine residues which are oxidized may be Met14 and Met125.

Oxidized methionine residues can be detected by reduced peptide mapping comprising reduction and digestion of the protein and liquid chromatography coupled to mass spectrometry (LC-MS). One suitable example of reduced peptide mapping is described in more detail in the examples below. The percentage of oxidized methionine residues can be calculated by dividing the number of oxidized methionine residues by the total number of methionine residues within the sample analyzed. The oxidized methionine residues can also be detected by reversed-phase HPLC (RP-HPLC), since the oxidation of methionine alters the hydrophilicity of the protein.

The process of the present invention leads to a reduction of the percentage of oxidized methionine residues in the recombinant protein by at least 20%, preferably by at least 25% or 30%, more preferably by at least 35% or 40%, even more preferably by at least 45% or 48% and most preferably by at least 50% or 53%.

The term “recombinant protein” refers to any protein which can be produced by mammalian cell culture as the result of the transcription and translation of a gene encoding said recombinant protein which gene is carried on a recombinant nucleic acid molecule that has been introduced into the mammalian host cell. The recombinant protein may not be produced naturally in the mammalian cells used or the recombinant protein may be produced naturally in the mammalian cells used, but at a lower level so that the recombinant cell can be distinguished from the non-modified, non-recombinant cell by the higher expression level of the recombinant protein compared to the expression level in a non-modified, non-recombinant cell. Preferably, the recombinant protein is not produced naturally by the mammalian cell so that the recombinant cell can be distinguished from the non-modified, non-recombinant cell by the expression of the recombinant protein which is detectable in the recombinant cell by methods known to the skilled person, but which is not detectable in the non-modified, non-recombinant cell.

In particular, the term “recombinant protein” encompasses therapeutic proteins such as cytokines, growth factors, clotting factors, vaccines and antibodies. Preferably, the recombinant protein is an Fc containing protein such as an antibody or a fusion protein of the Fc portion of an IgG antibody with parts or all of another protein.

In one embodiment, the recombinant protein may be a recombinant antibody. The term “recombinant antibody” refers to any antibody which can be produced by mammalian cell culture as the result of the transcription and translation of a gene encoding said recombinant antibody which gene is carried on a recombinant nucleic acid molecule that has been introduced into the mammalian host cell. The recombinant antibody may not be produced naturally in the mammalian cells used or the recombinant antibody may be produced naturally in the mammalian cells used, but at a lower level so that the recombinant cell can be distinguished from the non-modified, non-recombinant cell by the higher expression level of the recombinant antibody compared to the expression level in a non-modified, non-recombinant cell.

Preferably, the recombinant antibody is not produced naturally by the mammalian host cell used for its production, so that the recombinant cell can be distinguished from the non-modified, non-recombinant cell by the expression of the recombinant antibody which is detectable in the recombinant cell by methods known to the skilled person, but which is not detectable in the non-modified, non-recombinant cell.

The terms “immunoglobulin” and “antibody” are used interchangeably herein. The immunoglobulin may be a monoclonal antibody, multispecific antibody (e.g. bispecific antibody) or fragments thereof exhibiting the desired antigen binding activity. Naturally occurring antibodies are molecules with varying structures. For example, native IgG antibodies are heterotetrameric glycoproteins of about 150,000 Daltons, composed of two identical light chains and two identical heavy chains that are linked by disulfide bonds. From N- to C-terminus, each heavy chain has a variable domain (VH), also called a variable heavy domain or a heavy chain variable domain followed by three or four constant domains (CH1, CH2, CH3 and optionally CH4). Similarly, from N- to C-terminus, each light chain has a variable domain (VL), also called a variable light domain or a light chain variable domain followed by a constant light chain (CL) domain. The light chain of an antibody may be assigned to one of two types, called kappa (κ) and lambda (λ), based on the amino acid sequence of its constant domain.

“Antibody fragments” comprise a portion of a full-length antibody, generally the antigen binding or variable region thereof. Examples of antibody fragments include Fab, Fab′, F(ab′)2, and Fv fragments, single-chain antibody molecules, diabodies, linear antibodies and multispecific antibodies formed from antibody fragments.

Preferably, the immunoglobulin is a monoclonal antibody. The term “monoclonal antibody” as used herein refers to an antibody obtained from a population of substantially homogeneous antibodies, i.e. the individual antibodies of the population are identical except for possible naturally occurring mutations that may be present in minor amounts. In contrast to polyclonal antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method.

The immunoglobulin may be of the murine classes IgG1, IgG2a, IgG2b, IgM, IgA, IgD or IgE, the human classes IgG1, IgG2, IgG3, IgG4, IgM, IgA1, IgA2, IgD or IgE, or combinations or fragments thereof.

The immunoglobulin may recognize any one or a combination of proteins including, but not limited to the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, CD152, IL-1a, IL-1ß, IL-1, IL-2, IL-3, IL-7, IL-4, IL-5, IL-8, IL-10, IL-12, IL-23, IL-2 receptor, IL-4 receptor, IL-6 receptor, IL-12 receptor, IL-13 receptor, IL-18 receptor subunits, PDGF-β, and analogues thereof, PLGF, VEGF, TGF, TGF-β2, TGF-p1, EGF receptor, PLGF receptor, VEGF receptor, platelet receptor gpIlb/IIIa, thrombopoietin receptor, apoptosis receptor PD-1, hepatocyte growth factor, osteoprotegerin ligand, interferon gamma, B lymphocyte stimulator BLyS, T-cell activation regulator CTLA-4, C5 complement, IgE, tumor antigen CA125, tumor antigen MUC1, PEM antigen, ErbB2/HER-2, tumor-associated epitopes that are present in elevated levels in the sera of patients, cancer-associated epitopes or proteins expressed on breast, colon, squamous cell, prostate, pancreatic, lung and/or kidney cancer cells and/or on melanoma, glioma, or neuroblastoma cells, the necrotic core of a tumor, integrin alpha 4 beta 7, the integrin VLA-4, B2 integrins, α4β1 and α4β7 integrin, TRAIL receptors 1,2,3, and 4, RANK, a RANK ligand (RANKL), TNF-α, the adhesion molecule VAP-1, epithelial cell adhesion molecule (EpCAM), intercellular adhesion molecule-3 (ICAM-3), leukointegrin adhesin, cardiac myosin heavy chain, parathyroid hormone, sclerostin, MHC I, carcinoembryonic antigen (CEA), alpha-fetoprotein (AFP), tumor necrosis factor (TNF), Fc-y-1 receptor, HLA-DR 10 beta, HLA-DR antigen, L-selectin, and IFN-γ.

The immunoglobulin may for example be afelimomab, abciximab, adalimumab, alemtuzumab, arcitumomab, belimumab, canakinumab, cetuximab, denosumab, trastuzumab, imciromab, capromab, infliximab, ipilimumab, abciximab, rituximab, basiliximab, palivizumab, natalizumab, nivolumab, nofetumomab, omalizumab, daclizumab, ibritumomab, muromonab-CD3, edrecolomab, gemtuzumab, golimumab, certolizumab, eculizumab, ustekinumab, ocrelizumab, ofatumumab, obinutuzumab, panitumumab, pertuzumab, ranibizumab, romosozumab, tocilizumab, tositumomab, clenoliximab, keliximab, galiximab, foravirumab, lexatumumab, bevacizumab, satumomab, catumaxomab, brentuximab, raxibazumab, tosimumab, efalizumab, motavizumab, bezlotoxumab, ixekizumab, olaratumab, pembrolizumab, secukinumab and vedolizumab.

The immunoglobulin is preferably an IgG molecule, such as an IgG1, IgG2, IgG3, or IgG4 molecule. More preferably, the immunoglobulin is IgG1 or IgG4. Even more preferably, the immunoglobulin is an IgG1 wherein at least the Fc part is human.

The immunoglobulin may be a murine-human chimeric IgG1 wherein the Fc part of the IgG1 is human and the variable region is of mouse origin. Alternatively, the immunoglobulin may be a humanized IgG1 or IgG4 form of a murine progenitor, meaning that the CDRs of the variable domain are derived from mouse and the framework regions of the variable domain are derived from human. Alternatively, the immunoglobulin may be a fully human IgG1 antibody, i.e. an antibody in which all parts are derived from human origin.

Preferably, the recombinant protein is a fusion protein of the Fc portion of an IgG antibody with parts or all of another protein. Such fusion proteins include, but are not limited to, etanercept and lenercept (fusion with TNF receptor), aflibercept and conbercept (fusion with extracellular domains of VEGF receptors 1 and 2), abatacept and belatacept (fusion with extracellular domain of CTLA-4) and rilonacept (fusion with extracellular portions of the human interleukin-1 receptor component (IL-1R1) and IL-1 receptor accessory protein (IL-1RAcP)). More preferably, the recombinant protein is a fusion protein of the Fc region of an IgG antibody with the extracellular domains of VEGF receptors 1 and 2 and most preferably, the recombinant protein is aflibercept.

Aflibercept is presently marketed under the names Eylea® and Zaltrap® and is also known as VEGF-trap. It is a recombinant human soluble VEGF receptor fusion protein in which the second immunoglobulin-like domain of VEGF receptor 1 and the third immunoglobulin-like domain of VEGF receptor 2 are fused to the Fc portion of human IgG1 (Holash et al. (2002) Proc. Natl. Acad. Sci. USA 99(17): 11393-11398; WO 00/75319 A1). The CAS number of aflibercept is 862111-32-8. Eylea® has received a marketing authorization for the treatment of wet age-related macular degeneration, visual impairment due to diabetic macular oedema (DME) and diabetic retinopathy in patients with diabetic macular edema. Zaltrap® has received a marketing authorization for the treatment of adults with metastatic colorectal cancer (MCRC) that is resistant to or has progressed after an oxaliplatin-containing regimen in combination with irinotecan/5-fluorouracil/folinic acid (FOLFIRI) chemotherapy.

In the method of the present invention the recombinant protein is produced in eukaryotic cells and in particular in mammalian cells. Suitable mammalian cells for expressing the recombinant protein in the method of the present invention include Chinese Hamster Ovary (CHO) cells, NS0 myeloma cells, COS cells, SP2/0 cells, monkey kidney CV1, human embryonic kidney cell (293), baby hamster kidney cells (BHK), mouse Sertoli cells (TM4), African green monkey kidney cells (VERO-76), human cervical carcinoma cells (HeLa), canine kidney cells (MDC), buffalo rat liver cells (BRL 3 A), human lung cells (W138), human liver cells (Hep G2), mouse mammary tumor cells (MMT 060562), TRI cells, MRC 5 cells and FS4 cells.

Preferably, the mammalian cells are derived from a rodent selected from hamster and mouse.

More preferably, the mammalian cells are Chinese hamster ovary (CHO) cells such as CHO-K1, CHO-DUKX (dihydrofolate reductase (DHFR)-deficient CHO cells), CHO-pro3 (proline-dependent cells) and CHO-DG44 (dihydrofolate reductase (DHFR)-deficient and proline-dependent cells).

Even more preferably the cells are CHO-K1 cells. The CHO-K1 cell line has been obtained from a single clone of the original CHO cells (Kao and Puck (1968) Proc. Nat. Acad. Sci. USA 60(4): 1275-1281). The CHO-K1 cell line can be adapted to suspension growth and/or to a chemically defined medium (see, e.g., Bort et al. (2010) Biotechnol. J. 5(10): 1090-1097). In the present invention preferably CHO-K1 cells or cells derived therefrom are used. The cells which are derived from the CHO-K1 cells are cells which originate from the CHO-K1 cells, but have been subjected to one or more adaptation processes, such as adaptation to serum-free medium or suspension growth.

The mammalian cells have been transformed, i.e. genetically modified, with at least one recombinant nucleic acid molecule such as an expression vector which enables the stable production of the recombinant protein in the mammalian host cells. Due to the presence of the recombinant nucleic acid molecule the transformed or recombinant cells can be distinguished from untransformed or non-recombinant cells.

The elements and methods needed to construct expression vectors which are suitable for expressing a recombinant protein such as a Fc fusion protein in mammalian cells, preferably in CHO cells, are well-known to the skilled person and described for example in Makrides et al. (1999) Protein Expr. Purif. 17: 183-202 and Kaufman (2000) Mol. Biotechnol. 16: 151-161. Further, the skilled person is aware of methods for introducing the expression vectors into the mammalian cells. These methods include the use of commercially available transfection kits such as Lipofectamine® of ThermoFisher, PElmax of Polyplus Sciences) or Freestyle Max of Invitrogen. Further suitable methods include electroporation, calcium phosphate-mediated transfection and DEAE-dextrane transfection. After transfection the cells are subjected to selection by treatment with a suitable agent based on the selection marker(s) encoded by the expression vector(s) to identify the stably transfected cells which contain the recombinant nucleic acid molecule.

The terms “medium”, “cell culture medium” and “culture medium” are interchangeably used herein and refer to a solution containing nutrients which are required for growing mammalian cells. Typically, a cell culture medium provides essential and non-essential amino acids, vitamins, energy sources, lipids, and trace elements required by the cell for minimal growth and/or survival. Preferably, the medium is chemically defined in that all its components and their concentration are known. Also preferably, the medium is serum-free and hydrolysate-free and does not contain any components derived from animals. In a more preferred embodiment the medium is serum-free and hydrolysate-free and does not contain any components derived from animals or insulin.

In one embodiment the medium used in the method of the present invention is a commercially available medium such as PolCHO P Powder Base CD, ActiPro (both available from GE), PowerCHO-2, ProCHO-5 (both available from Lonza) or EX-CELL® Advanced CHO fed batch medium (available from Sigma).

For culturing the mammalian cells different strategies are available, including batch culture, perfusion culture, continuous culture and fed-batch culture. Within the method of the present invention, a fed-batch culture process is used. In fed-batch culture the culturing process is started with a certain volume of the basal medium and one or more feed media comprising one or more nutrients are fed at later time-point(s) of the culture process to prevent nutrient depletion while no product is removed from the cell culture broth. Accordingly, the term “feeding” means that at least one component is added to an existing culture of cells.

The term “basal medium” is intended to refer to the medium which is used from the beginning of the cell culture process. The mammalian cells are inoculated into the basal medium and grown in this medium for a certain period until the feeding is started. The basal medium meets the definition of the culture medium as provided above. If a commercially available medium is used, additional components may be added to the basal medium.

The feed medium is added to the cell culture after the cells have been cultured in the basal medium for a certain period. The feed medium serves to prevent nutrient depletion and therefore may not have the same composition as the basal medium. In particular, the concentration of one or more nutrients may be higher in the feed medium than in the basal medium. In one embodiment, the feed medium has the same composition as the basal medium. In another embodiment, the feed medium has another composition as the basal medium. The feed medium may be added continuously or as a bolus at defined time points. In a preferred embodiment the feed medium is added as a bolus at defined time points.

Suitable feed media are known to the skilled person and include PolCHO Feed-A Powder Base CD, PolCHO Feed-B Powder Base CD, Cell Boost 7a and Cell Boost 7b (all available from GE), BalanCD® CHO Feed 3 Medium (available from Irvine Scientific) and EX-CELL® Advanced CHO feed 1 (available from Sigma). The feed medium may comprise additional components such as N-acetylglucosamine, manganese ions and galactose.

Preferably, EX-CELL® Advanced CHO fed batch medium is used as a basal medium and EX-CELL® Advanced CHO feed 1 is used as feed medium.

If in the context of the present invention reference is made to “days of culture”, this is intended to refer to the days after inoculation of the cells into the basal cell culture medium.

In the method of the present invention the feeding with a feed medium as described above preferably starts on day 2 to 4 of the cell culture, i.e. 2 to 4 days after inoculation of the cells into the basal medium. More preferably, the feeding with a feed medium as described above starts on day 3 of the cell culture, i.e. 3 days after inoculation of the cells into the basal medium.

The cells may be fed with the feed medium one or more times, such as 1 to 10 times, preferably 2 to 9 times, more preferably 3 to 8 times, even more preferably 4 to 7 times and most preferably 5 times.

In one embodiment the cells are fed with the feed medium at days 3, 5, 7, 9 and 11 of the cell culture, i.e. 3, 5, 7, 9 and 11 days after inoculation of the cells into the basal medium.

The method of the present invention comprises feeding the cells with glutamine. This means that in addition to feeding the cells with a suitable feed medium as described above, glutamine is added to the cells during the feeding phase of the culture. The feeding phase starts with the first feeding of the feed medium as described above. The amino acid glutamine has been reported to be a significant nitrogen and energy source during mammalian cell culture and is therefore one of the most consumed amino acids in mammalian cell culture. Accordingly, it is necessary to supplement the cells with high levels of glutamine to achieve a high specific growth rate, cell density and productivity. However, so far it had not been shown that the feeding with glutamine also affects protein degradation pathways and in particular the oxidation of methionine residues within a recombinant protein.

The concentration of glutamine used for feeding the cells is 0.2 mM to 5 mM, preferably 0.3 mM to 4 mM, more preferably 0.4 mM to 3 mM and most preferably 0.5 mM to 2 mM.

In one embodiment, the concentration of glutamine used for feeding the cells is 0.2 mM to 5 mM, preferably 0.5 mM to 4 mM, more preferably 1 mM to 3 mM and even more preferably it is 2 mM and the cells are fed with glutamine on day 3 to 5 of the culture. In one embodiment, the concentration of glutamine used for feeding the cells is 0.2 mM to 5 mM, preferably 0.5 mM to 4 mM, more preferably 1 mM to 3 mM and even more preferably it is 2 mM and the cells are fed with glutamine on day 4 of the culture. Most preferably, the cells are fed with 2 mM glutamine on day 4 of the culture.

The method of the present invention may comprise feeding the cells with glutamine more than once, i.e. the method of the present invention may comprise more than one feeding step with glutamine. The method of the present invention may comprise feeding the cells with glutamine one to six times, preferably two to five times and most preferably three times. The glutamine concentration used for feeding the cells may be the same in all feeding steps. Alternatively and preferably, the glutamine concentration used for feeding the cells may differ between the feeding steps. More preferably, the glutamine concentration in the first feeding step is higher than the glutamine concentration in the subsequent feeding steps. Most preferably, the method comprises three feeding steps with glutamine and the glutamine concentration in the first feeding step is higher than the glutamine concentration in the second and third feeding steps.

In one embodiment the method of the present invention comprises feeding the cells with glutamine two to five times, wherein the glutamine concentration in the first feeding step is as described above and the glutamine concentration in the second to fifth feeding step is 0.2 mM to 2 mM, preferably 0.3 mM to 1.8 mM, more preferably 0.4 mM to 1.4 mM and most preferably 0.5 mM to 1 mM. The second feeding step is on day 6 to 8 of the culture, preferably on day 7 of the culture. The third feeding step is on day 9 to 11 of the culture, preferably on day 10 of the culture.

In one embodiment the method of the present invention comprises feeding the cells with glutamine three times, wherein the glutamine concentration in the first feeding step is as described above and the glutamine concentration in the second and third feeding step is 0.2 mM to 2 mM, preferably 0.3 mM to 1.8 mM, more preferably 0.4 mM to 1.4 mM and most preferably 0.5 mM to 1 mM. The second feeding step is on day 6 to 8 of the culture, preferably on day 7 of the culture. The third feeding step is on day 9 to 11 of the culture, preferably on day 10 of the culture.

In one preferred embodiment of the present invention the method comprises feeding the cells with 2 mM glutamine on day 4 of the culture, with 0.5 or 1.0 mM glutamine on day 7 of the culture and with 0.5 or 1.0 mM glutamine on day 10 of the culture.

Accordingly, the present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture; and
(c) obtaining the aflibercept.

The method of the present invention may additionally comprise feeding the cells with methionine. It has been shown that culturing cells in a medium to which methionine was added leads to a reduction of the percentage of oxidized forms of recombinant FSH (WO 2007/003640 A1). The present inventors have shown that feeding the cells expressing the recombinant protein with methionine in addition to the feeding with glutamine further reduces the percentage of oxidized methionine residues within the recombinant protein.

Accordingly, the present invention is directed to a method for reducing the percentage of oxidized methionine residues in a recombinant protein, said method comprising culturing cells expressing said recombinant protein in a suitable cell culture medium and feeding the cells with glutamine and methionine.

The present invention is also directed to a method for producing a recombinant protein, comprising
(a) culturing cells expressing the recombinant protein in a suitable cell culture medium;
(b) feeding the cells with glutamine and methionine; and
(c) obtaining the recombinant protein.

The present invention is also directed to a method for producing aflibercept, comprising
(a) culturing cells expressing aflibercept in a suitable cell culture medium;
(b) feeding the cells with glutamine and methionine; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 0.2 mM to 5 mM glutamine and methionine.

The present invention is also directed to a method for producing a recombinant protein, comprising
(a) culturing cells expressing the recombinant protein in a suitable cell culture medium;
(b) feeding the cells with 0.2 mM to 5 mM glutamine and methionine; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and methionine; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and feeding the cells with methionine.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and feeding the cells with methionine; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and feeding the cells with methionine; and
(c) obtaining the aflibercept.

The concentration of methionine used for feeding the cells is 250 mg/l to 1000 mg/1, preferably 300 mg/l to 800 mg/1, more preferably 400 mg/l to 600 mg/l and most preferably it is 500 mg/l.

Accordingly, the present invention is directed to a method for reducing the percentage of oxidized methionine residues in a recombinant protein, said method comprising culturing cells expressing said recombinant protein in a suitable cell culture medium and feeding the cells with glutamine and 500 mg/l methionine.

The present invention is also directed to a method for producing a recombinant protein, comprising
(a) culturing cells expressing the recombinant protein in a suitable cell culture medium;
(b) feeding the cells with glutamine and 500 mg/l methionine; and
(c) obtaining the recombinant protein.

The present invention is also directed to a method for producing aflibercept, comprising
(a) culturing cells expressing aflibercept in a suitable cell culture medium;
(b) feeding the cells with glutamine and 500 mg/l methionine; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 0.2 mM to 5 mM glutamine and 500 mg/l methionine.

The present invention is also directed to a method for producing a recombinant protein, comprising
(a) culturing cells expressing the recombinant protein in a suitable cell culture medium;
(b) feeding the cells with 0.2 mM to 5 mM glutamine and 500 mg/l methionine; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and 500 mg/l methionine; and
(c) obtaining the aflibercept.

The present invention refers to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and feeding the cells with 500 mg/l methionine.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and feeding the cells with 500 mg/l methionine; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and feeding the cells with 500 mg/l methionine; and
(c) obtaining the aflibercept.

In one embodiment, the concentration of methionine used for feeding the cells is 250 mg/l to 1000 mg/1, preferably 300 mg/l to 800 mg/1, more preferably 400 mg/l to 600 mg/l and most preferably it is 500 mg/l and the cells are fed with methionine on day 8 to 12 of the culture. In one embodiment, the concentration of methionine used for feeding the cells is 250 mg/l to 1000 mg/1, preferably 300 mg/l to 800 mg/1, more preferably 400 mg/l to 600 mg/l and most preferably it is 500 mg/l and the cells are fed with methionine on day 7 to 11 of the culture. In one embodiment, the concentration of methionine used for feeding the cells is 250 mg/l to 1000 mg/1, preferably 300 mg/l to 800 mg/1, more preferably 400 mg/l to 600 mg/l and most preferably it is 500 mg/l and the cells are fed with methionine on day 9 or 10 of the culture. Most preferably, the cells are fed with 500 mg/l methionine on day 9 of the culture.

Accordingly, the present invention is directed to a method for reducing the percentage of oxidized methionine residues in a recombinant protein, said method comprising culturing cells expressing said recombinant protein in a suitable cell culture medium and feeding the cells with glutamine and 500 mg/l methionine on day 9 of the culture.

The present invention is also directed to a method for producing a recombinant protein, comprising
(a) culturing cells expressing the recombinant protein in a suitable cell culture medium;
(b) feeding the cells with glutamine and 500 mg/l methionine on day 9 of the culture; and
(c) obtaining the recombinant protein.

The present invention is also directed to a method for producing aflibercept, comprising
(a) culturing cells expressing aflibercept in a suitable cell culture medium;
(b) feeding the cells with glutamine and 500 mg/l methionine on day 9 of the culture; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 0.2 mM to 5 mM glutamine and 500 mg/l methionine on day 9 of the culture.

The present invention is also directed to a method for producing a recombinant protein, comprising
(a) culturing cells expressing the recombinant protein in a suitable cell culture medium;
(b) feeding the cells with 0.2 mM to 5 mM glutamine and 500 mg/l methionine on day 9 of the culture; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and 500 mg/l methionine on day 9 of the culture; and
(c) obtaining the aflibercept.

The present invention refers to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and feeding the cells with 500 mg/l methionine on day 9 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and feeding the cells with 500 mg/l methionine on day 9 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and feeding the cells with 500 mg/l methionine on day 9 of the culture; and
(c) obtaining the aflibercept.

The method of the present invention may comprise feeding the cells with methionine more than once, i.e. the method of the present invention may comprise more than one feeding step with methionine. The method of the present invention may comprise feeding the cells with methionine one to four times, preferably one to three times and most preferably one or two times. The methionine concentration used for feeding the cells may be the same in all feeding steps. Alternatively, the methionine concentration used for feeding the cells differs between the feeding steps. Preferably, the methionine concentration used for feeding the cells is the same in all feeding steps.

In one embodiment the method of the present invention comprises feeding the cells with methionine two times, wherein the methionine concentration in the first and the second feeding step is 250 mg/l to 1000 mg/1, preferably 300 mg/l to 800 mg/1, more preferably 400 mg/l to 600 mg/l and most preferably it is 500 mg/l. The first feeding step with methionine is on day 8 to 10 of the culture, preferably on day 9 of the culture. The second feeding step is on day 9 to 11 of the culture, preferably on day 10 of the culture.

In one preferred embodiment of the present invention the method comprises feeding the cells with 2 mM glutamine on day 4 of the culture, with 0.5 or 1.0 mM glutamine on day 7 of the culture, with 0.5 or 1.0 mM glutamine on day 10 of the culture, with 500 mg/l methionine on day 9 of the culture and with 500 mg/l methionine on day 10 of the culture.

In one embodiment glutamine is the only amino acid used for feeding the cells. This means that only glutamine is used for feeding the cells separately from the feed medium. However, the feed medium may comprise amino acids other than glutamine.

In another embodiment glutamine and methionine are the only amino acids used for feeding the cells. This means that only glutamine and methionine are used for feeding the cells separately from the feed medium. However, the feed medium may comprise amino acids other than glutamine and methionine.

In another embodiment the cells are not fed with asparagine separately from the feed medium. However, the feed medium may comprise asparagine.

The method of the present invention may be performed at a constant temperature, e.g. at a temperature of 37° C.±0.2° C. Alternatively, the method of the present invention may comprise reducing the culture temperature from a first temperature to a second temperature, i.e. the temperature is actively downregulated. Hence, the second temperature is lower than the first temperature. The first temperature may be 37° C.±0.2° C. The second temperature may be in the range of from 30° C. to 36° C., preferably it is in the range of 31° C. to 35° C., more preferably it is in the range of 32° C. to 35° C., and most preferably it is 34° C. The reduction of the culture temperature from the first temperature to the second temperature may be performed on day 5 to 7 of the culture, preferably on day 6 of the culture.

Accordingly, the present invention is directed to a method for reducing the percentage of oxidized methionine residues in a recombinant protein, said method comprising culturing cells expressing said recombinant protein in a suitable cell culture medium, feeding the cells with glutamine and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with glutamine and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with glutamine and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with 0.2 mM to 5 mM glutamine and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with glutamine and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with glutamine and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with glutamine and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with 0.2 mM to 5 mM glutamine and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with glutamine and reducing the culture temperature from a first temperature of 37° C. to a second temperature of 34° C. on day 6 of the culture.

The present invention is also directed to a method for producing a recombinant protein, comprising
(a) culturing cells expressing the recombinant protein in a suitable cell culture medium;
(b) feeding the cells with glutamine and reducing the culture temperature from a first temperature of 37° C. to a second temperature of 34° C. on day 6 of the culture; and
(c) obtaining the recombinant protein.

The present invention is also directed to a method for producing aflibercept, comprising
(a) culturing cells expressing aflibercept in a suitable cell culture medium;
(b) feeding the cells with glutamine and reducing the culture temperature from a first temperature of 37° C. to a second temperature of 34° C. on day 6 of the culture; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and reducing the culture temperature from a first temperature of 37° C. to a second temperature of 34° C. on day 6 of the culture.

The present invention is also directed to a method for producing a recombinant protein, comprising
(a) culturing cells expressing the recombinant protein in a suitable cell culture medium;
(b) feeding the cells with 0.2 mM to 5 mM glutamine and reducing the culture temperature from a first temperature of 37° C. to a second temperature of 34° C. on day 6 of the culture; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and reducing the culture temperature from a first temperature of 37° C. to a second temperature of 34° C. on day 6 of the culture; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C. on day 6 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C. on day 6 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C. on day 6 of the culture; and
(c) obtaining the aflibercept.

The method of the present invention may comprise a second step of reducing the temperature from the second temperature to a third temperature, i.e. the temperature is actively downregulated. Hence, the third temperature is lower than the second temperature. The third temperature may be in the range of 31° C. to 33° C., even more preferably it is 32° C. The reduction of the culture temperature from the second temperature to the third temperature may be performed on day 9 to 12 of the culture, preferably on day 10 of the culture.

The present invention is directed to a method for reducing the percentage of oxidized methionine residues in a recombinant protein, said method comprising culturing cells expressing said recombinant protein in a suitable cell culture medium, feeding the cells with glutamine and reducing the culture temperature from a first temperature to a second temperature and from a second temperature to a third temperature.

The present invention is also directed to a method for producing a recombinant protein, comprising
(a) culturing cells expressing the recombinant protein in a suitable cell culture medium;
(b) feeding the cells with glutamine and reducing the culture temperature from a first temperature to a second temperature and from a second temperature to a third temperature; and
(c) obtaining the recombinant protein.

The present invention is also directed to a method for producing aflibercept, comprising
(a) culturing cells expressing aflibercept in a suitable cell culture medium;
(b) feeding the cells with glutamine and reducing the culture temperature from a first temperature to a second temperature and from a second temperature to a third temperature; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and reducing the culture temperature from a first temperature to a second temperature and from a second temperature to a third temperature.

The present invention is also directed to a method for producing a recombinant protein, comprising
(a) culturing cells expressing the recombinant protein in a suitable cell culture medium;
(b) feeding the cells with 0.2 mM to 5 mM glutamine and reducing the culture temperature from a first temperature to a second temperature and from a second temperature to a third temperature; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and reducing the culture temperature from a first temperature to a second temperature and from a second temperature to a third temperature; and
(c) obtaining the aflibercept.

Accordingly, the present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture; and
reducing the culture temperature from a first temperature to a second temperature and from a second temperature to a third temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature to a second temperature and from a second temperature to a third temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature to a second temperature and from a second temperature to a third temperature; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with glutamine and reducing the culture temperature from a first temperature of 37° C. to a second temperature of 34° C. and from a second temperature of 34° C. to a third temperature of 32° C.

The present invention is also directed to a method for producing a recombinant protein, comprising
(a) culturing cells expressing the recombinant protein in a suitable cell culture medium;
(b) feeding the cells with glutamine and reducing the culture temperature from a first temperature of 37° C. to a second temperature of 34° C. and from a second temperature of 34° C. to a third temperature of 32° C.; and
(c) obtaining the recombinant protein.

The present invention is also directed to a method for producing aflibercept, comprising
(a) culturing cells expressing aflibercept in a suitable cell culture medium;
(b) feeding the cells with glutamine and reducing the culture temperature from a first temperature of 37° C. to a second temperature of 34° C. and from a second temperature of 34° C. to a third temperature of 32° C.; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and reducing the culture temperature from a first temperature of 37° C. to a second temperature of 34° C. and from a second temperature of 34° C. to a third temperature of 32° C.

The present invention is also directed to a method for producing a recombinant protein, comprising
(a) culturing cells expressing the recombinant protein in a suitable cell culture medium;
(b) feeding the cells with 0.2 mM to 5 mM glutamine and reducing the culture temperature from a first temperature of 37° C. to a second temperature of 34° C. and from a second temperature of 34° C. to a third temperature of 32° C.; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and reducing the culture temperature from a first temperature of 37° C. to a second temperature of 34° C. and from a second temperature of 34° C. to a third temperature of 32° C.; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture; and
reducing the culture temperature from a first temperature of 37° C. to a second temperature of 34° C. and from a second temperature of 34° C. to a third temperature of 32° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C. and from a second temperature of 34° C. to a third temperature of 32° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C. and from a second temperature of 34° C. to a third temperature of 32° C.; and
(c) obtaining the aflibercept.

In a preferred embodiment the method of the present invention comprises reducing the culture temperature from a first temperature of 37° C. to a second temperature of 34° C. on day 6 of the culture and from the second temperature of 34° C. to a third temperature of 32° C. on day 10 of the culture.

The present invention is directed to a method for reducing the percentage of oxidized methionine residues in a recombinant protein, said method comprising culturing cells expressing said recombinant protein in a suitable cell culture medium, feeding the cells with glutamine and reducing the culture temperature from a first temperature of 37° C. to a second temperature of 34° C. on day 6 of the culture and from the second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with glutamine and reducing the culture temperature from a first temperature of 37° C. to a second temperature of 34° C. on day 6 of the culture and from the second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with glutamine and reducing the culture temperature from a first temperature of 37° C. to a second temperature of 34° C. on day 6 of the culture and from the second temperature of 34° C. to a third temperature of 32° C. on day 10 of the culture; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and reducing the culture temperature from a first temperature of 37° C. to a second temperature of 34° C. on day 6 of the culture and from the second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 0.2 mM to 5 mM glutamine and reducing the culture temperature from a first temperature of 37° C. to a second temperature of 34° C. on day 6 of the culture and from the second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and reducing the culture temperature from a first temperature of 37° C. to a second temperature of 34° C. on day 6 of the culture and from the second temperature of 34° C. to a third temperature of 32° C. on day 10 of the culture; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture; and
reducing the culture temperature from a first temperature of 37° C. to a second temperature of 34° C. on day 6 of the culture and from a second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the aflibercept.

Accordingly, the present invention is directed to a method for reducing the percentage of oxidized methionine residues in a recombinant protein, said method comprising culturing cells expressing said recombinant protein in a suitable cell culture medium, feeding the cells with glutamine and methionine and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with glutamine and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with glutamine and methionine and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and methionine and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with 0.2 mM to 5 mM glutamine and methionine and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and methionine and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with glutamine and methionine and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with glutamine and methionine and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with glutamine and methionine and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium, feeding the cells with 0.2 mM to 5 mM glutamine and methionine and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with 0.2 mM to 5 mM glutamine and methionine and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with 0.2 mM to 5 mM glutamine and methionine and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and and with methionine and reducing the culture temperature from a first temperature of 37° C. to a second temperature of 34° C. on day 6 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C. on day 6 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C. on day 6 of the culture; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with methionine; and
reducing the culture temperature from a first temperature to a second temperature and from a second temperature to a third temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature to a second temperature and from a second temperature to a third temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature to a second temperature and from a second temperature to a third temperature; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with methionine and
reducing the culture temperature from a first temperature of 37° C. to a second temperature of 34° C. and from a second temperature of 34° C. to a third temperature of 32° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C. and from a second temperature of 34° C. to a third temperature of 32° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C. and from a second temperature of 34° C. to a third temperature of 32° C.; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with methionine; and
reducing the culture temperature from a first temperature of 37° C. to a second temperature of 34° C. on day 6 of the culture and from a second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C. on day 6 of the culture and from a second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium; (b) feeding the cells with 2 mM glutamine on day 4 of the culture, with 0.5 or 1.0 mM glutamine on day 7 of the culture and with 0.5 or 1.0 mM glutamine on day 10 of the culture and with methionine and reducing the culture temperature from a first temperature of 37° C. to a second temperature of 34° C. on day 6 of the culture and from a second temperature of 34° C. to a third temperature of 32° C. on day 10 of the culture; and
(c) obtaining the aflibercept.

Accordingly, the present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C. on day 6 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C. on day 6 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C. on day 6 of the culture; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine; and
reducing the culture temperature from a first temperature to a second temperature and from a second temperature to a third temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature to a second temperature and from a second temperature to a third temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature to a second temperature and from a second temperature to a third temperature; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and
reducing the culture temperature from a first temperature of 37° C. to a second temperature of 34° C. and from a second temperature of 34° C. to a third temperature of 32° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C. and from a second temperature of 34° C. to a third temperature of 32° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C. and from a second temperature of 34° C. to a third temperature of 32° C.; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine; and
reducing the culture temperature from a first temperature of 37° C. to a second temperature of 34° C. on day 6 of the culture and from a second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C. on day 6 of the culture and from a second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine and reducing the culture temperature from a first temperature of 37° C. to a second temperature of 34° C. on day 6 of the culture and from a second temperature of 34° C. to a third temperature of 32° C. on day 10 of the culture; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature to a second temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature to a second temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature to a second temperature; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C.; and
(c) obtaining the aflibercept.

The present invention is directed to a method for reducing the percentage of oxidized methionine residues in a recombinant protein, said method comprising culturing cells expressing said recombinant protein in a suitable cell culture medium and feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C. on day 6 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C. on day 6 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C. on day 6 of the culture; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture; and
reducing the culture temperature from a first temperature to a second temperature and from a second temperature to a third temperature.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature to a second temperature and from a second temperature to a third temperature; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature to a second temperature and from a second temperature to a third temperature; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and
reducing the culture temperature from a first temperature of 37° C. to a second temperature of 34° C. and from a second temperature of 34° C. to a third temperature of 32° C.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C. and from a second temperature of 34° C. to a third temperature of 32° C.; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C. and from a second temperature of 34° C. to a third temperature of 32° C.; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture; and
reducing the culture temperature from a first temperature of 37° C. to a second temperature of 34° C. on day 6 of the culture and from a second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 and day 10 of the culture; and
reducing the culture temperature from a first temperature of 37° C. to a second temperature of 34° C. on day 6 of the culture and from a second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 and day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 or 1.0 mM glutamine on day 7 of the culture and with 0.5 or 1.0 mM glutamine on day 10 of the culture and with 500 mg/l methionine on day 9 and day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 0.5 mM glutamine on day 7 of the culture and with 0.5 mM glutamine on day 10 of the culture and with 500 mg/l methionine on day 9 and day 10 of the culture; and
reducing the culture temperature from a first temperature of 37° C. to a second temperature of 34° C. on day 6 of the culture and from a second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 0.5 mM glutamine on day 7 of the culture and with 0.5 mM glutamine on day 10 of the culture and with 500 mg/l methionine on day 9 and day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 0.5 mM glutamine on day 7 of the culture and with 0.5 mM glutamine on day 10 of the culture and with 500 mg/l methionine on day 9 and day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the aflibercept.

The present invention is directed to a method for reducing the percentage of oxidized methionine residues in a recombinant protein, said method comprising:
culturing cells expressing said recombinant protein in a suitable cell culture medium;
feeding the cells with 2 mM glutamine on day 4 of the culture, with 1.0 mM glutamine on day 7 of the culture and with 1.0 mM glutamine on day 10 of the culture and with 500 mg/l methionine on day 9 and day 10 of the culture; and
reducing the culture temperature from a first temperature of 37° C. to a second temperature of 34° C. on day 6 of the culture and from a second temperature of 34° C. to a third temperature of 32° C. on day 10 of the culture.

The present invention is also directed to a method for producing a recombinant protein, comprising
(a) culturing cells expressing the recombinant protein in a suitable cell culture medium;
(b) feeding the cells with 2 mM glutamine on day 4 of the culture, with 1.0 mM glutamine on day 7 of the culture and with 1.0 mM glutamine on day 10 of the culture and with 500 mg/l methionine on day 9 and day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the recombinant protein.

The present invention is also directed to a method for producing aflibercept, comprising
(a) culturing cells expressing aflibercept in a suitable cell culture medium;
(b) feeding the cells with 2 mM glutamine on day 4 of the culture, with 1.0 mM glutamine on day 7 of the culture and with 1.0 mM glutamine on day 10 of the culture and with 500 mg/l methionine on day 9 and day 10 of the culture and reducing the culture temperature from a first temperature of 37° C. to a second temperature of 34° C. on day 6 of the culture and from a second temperature of 34° C. to a third temperature of 32° C. on day 10 of the culture; and
(c) obtaining the aflibercept.

In one embodiment the pH of the cell culture medium remains constant, meaning that the pH is not actively up- or down-regulated in the process and that always the same preset pH is used. Nevertheless, minor variations of the pH may occur during the culturing process. The pH of the cell culture medium is 7.1±0.3.

In the process of the present invention the cells are cultured under aerobic conditions, i.e. a level of dissolved oxygen of 5% to 95%, preferably of 30%.

If foaming of the cell culture occurs, antifoam agent may be added to the culture at any time during the process of the present invention.

The term “inoculation of the cells into the cell culture medium” refers to the step of contacting the cells with the cell culture medium under conditions which are suitable for growth and proliferation of the cells. It does not refer to any pre-culture of the cells which aims at expanding the cells until a certain number of cells is obtained. The cell culture medium used for inoculating the cells is the basal medium.

The method of the present invention may further comprise feeding glucose, if the concentration of glucose in the culture medium is determined to be below a certain threshold level. In particular, the method of the present invention may further comprise feeding glucose, if the concentration of glucose in the culture medium is below 4 g/l. The glucose is added to a concentration of 6 g/l in the culture medium.

In the method of the present invention the cells expressing the recombinant protein are cultured for a period of 12 to 18 days, preferably of 13 to 16 days and most preferably for a period of 14 days. The culture period is calculated from the time of inoculating the cells in the basal medium as the starting point and the time of harvesting the cells as the end point.

The method of the present invention does not substantially reduce the viability and/or productivity of the cells and does not substantially affect protein stability. In particular, in the method of the present invention the cell viability is reduced by less than 15%, preferably by less than 12%, more preferably by less than 10% and most preferably by less than 8%. Further, in the method of the present invention the maximum viable cell density is reduced by less than 15%, preferably by less than 12%, more preferably by less than 10% and most preferably by less than 8%. The cell number and viability can be determined using a cell counter and viability analyzer such as Vi-CELL Cell Counter & Cell Viability Analyzer (Beckmann).

In the method of the present invention the titer of the recombinant protein is reduced by less than 15%, preferably by less than 12%, more preferably by less than 10% and most preferably by less than 8%. The product titer can be determined by any method known to the skilled person, for example by Bio-Layer Interferometry (BLI).

One parameter to determine protein stability is the monomer content which can be determined using size exclusion chromatography. In the method of the present invention the monomer content does not substantially decrease, i.e. the monomer content is reduced by less than 10%, preferably by less than 7%, more preferably by less than 5% and most preferably by less than 3%.

After the recombinant protein has been produced according to any of the methods of the present invention for producing a recombinant protein, the recombinant protein is harvested from the cell culture. Since recombinant proteins, in particular Fc fusion proteins or antibodies, expressed from mammalian cells are typically secreted into the cell culture fluid during the cultivation process, the product harvest at the end of the cultivation process occurs by separating cell culture fluid comprising the recombinant protein from the cells. The cell separation method should be gentle to minimize cell disruption to avoid the increase of cell debris and release of proteases and other molecules that could affect the quality of the recombinant protein product. Usually, the harvesting of the cell culture fluid comprising the recombinant protein involves centrifugation and/or filtration, whereby the recombinant protein is present in the supernatant and the filtrate, respectively. Expanded bed adsorption chromatography is an alternative method to avoid centrifugation/filtration methods.

After harvesting the cell culture fluid comprising the recombinant protein the recombinant protein has to be purified from the cell culture fluid. The purification of recombinant proteins and in particular Fc fusion proteins or recombinant antibodies is usually accomplished by a series of standard techniques that can include chromatographic steps such as anion exchange chromatography, cation exchange chromatography, affinity chromatography, hydrophobic interaction chromatography, hydroxyapatite chromatography and size exclusion chromatography. Further, the purification process may comprise one or more ultra-, nano- or diafiltration steps.

Within the present invention the terms “obtaining the recombinant protein” or “obtaining the aflibercept” are intended to refer to the steps of harvesting the cell culture fluid and purifying the recombinant protein or aflibercept.

After purifying the recombinant protein it can be used to prepare a pharmaceutical composition. A pharmaceutical composition is a composition which is intended to be delivered to a patient for treating or preventing a disease or condition. In addition to the active agent, i.e. the recombinant protein or aflibercept, a pharmaceutical composition typically contains at least one pharmaceutically acceptable excipient. Pharmaceutically acceptable excipients are substances which do not interfere with the physiological activity of the recombinant protein and which stabilize the pharmaceutical composition and/or enhance solubility or decrease viscosity of the pharmaceutical composition. Typical pharmaceutically acceptable excipients for recombinant proteins include buffers, salts, sugars or sugar alcohols, amino acids and surface-active agents. For example, the commercially available Eylea® composition comprises 10 mM sodium phosphate, pH 6.2, 40 mM NaCl, 0.03% polysorbate 20 and 5% sucrose. An alternative Eylea® composition may comprise 10 mM L-histidine/histidine hydrochloride, pH 6.2 or 6.5, 40 mM NaCl, 5% sucrose and 0.03% polysorbate 20.

The process of the present invention improves the biosimilarity of a biosimilar therapeutic protein to its reference product, i.e. the marketed therapeutic protein. The process of the present invention improves the biosimilarity of a biosimilar aflibercept to its reference product which is marketed under the name Eylea®. A biosimilar therapeutic protein is a therapeutic protein which is marketed after the patent protection for the original product has expired and which has the same amino acid sequence as the original product, but may slightly differ in posttranslational modifications. Nevertheless, it shows a physiological effect which is identical to that of the original product. When an application for a marketing authorisation for a biosimilar of a marketed protein is filed, it has to be shown that the structure of the biosimilar protein is comparable to the reference product.

By using the methods of the present invention the amount of oxidized methionine residues in the recombinant protein is comparable to that of the reference product, thereby improving the biosimilarity compared to the protein which has been produced without feeding the cells with glutamine.

The following examples are provided for illustrative purposes. It is thus understood that the examples are not to be construed as limiting. The skilled person in the art will clearly be able to envisage further modifications of the principles laid out herein.

### Examples

The methods of the present invention are supported and illustrated by reference to the following non-limiting examples.

1. Methods Used for Analyzing the Recombinant Protein

1.1 Protein a Purification

Cell culture harvests were centrifuged at 4000 rpm for 20 minutes at room temperature (Multifuge 3s; Haraeus). Supernatants were filtered through a 0.45 μm and subsequently through a 0.22 μm disposable filter unit (Millipore 50 mL disposable vacuum filtration system).

Samples were purified with GraviTrap columns (rProtein A GraviTrap; GE Healthcare) at room temperature. Protein A purification was done by a step elution with 80% binding buffer (20 mM sodium phosphate, 0.15 M NaCl, pH 6.2) and 20% elution buffer (0.1 M sodium citrate, pH 3.0). Eluates were neutralized with 0.5 M sodium phosphate, pH 6.5 and dialyzed against formulation buffer (5% sucrose, 8.5 mM NaH₂PO₄, 1.5 mM Na₂HPO₄, 40 mM NaCl, 0.03% polysorbate 20, pH 6.2) overnight at 4° C.

After dialysis samples were filtered (Acrodisc Syringe Filter, 0.2 μm Supor; Pall) and concentrated to approximately 10 mg/mL by centrifugation (Eppendorf 5415D, Sigma 2-16K) with VivaSpin6 ultrafiltration units (MWCO: 10 kDa; Sartorius) at 4000 g (4° C.). Protein concentration was determined by NanoDrop device (NanoDrop 2000c, Thermo Scientific). Samples were stored at −80° C. until analytical testing.

1.2 SEC-HPLC

Protein A purified protein samples were loaded onto a TSKgel G3000SWXL, (Tosoh, 300×7.8 mm, 5 μm) column to detect the monomer content of the aflibercept protein.

The protein was eluted by isocratic elution using 0.02 M sodium phosphate (pH 6.0) and 0.8 M sodium chloride at a flow rate of 1.0 mL/min at 25° C. Eluted species were detected at a wavelength of 214 nm and displayed on a graph showing the concentration of the eluted species vs time. The elution profile showed a main peak with the non-aggregated protein and some further peaks of the protein representing higher molecular weight forms of the protein. The area of all peaks was determined. In the tables the percentage of peak area for the monomer in relation to the total peak area of the eluted species is shown.

1.3 Reduced Peptide Mapping

By reduced peptide mapping (PepMap red) the purity of the aflibercept protein with regard to methionine and tryptophane oxidation was analyzed after digestion with trypsin and liquid chromatography coupled to mass spectrometry (LC-MS).

After reduction and alkylation, the protein was submitted to enzymatic cleavage with trypsin. The resulting peptides were analyzed by RP-UPLC-MS. During chromatography the peptides were eluted by changing the mobile phase from highly polar (trifluoroacetic acid in water) to less polar (trifluoroacetic acid in acetonitrile) and analyzed by mass spectrometry (Xevo G2-XS QTOF). The peptide data was processed and compared with the theoretical protein sequence and a reference sample to detect oxidations.

Samples were diluted with denaturation buffer (50 mM Tris(hydroxymethyl)aminomethane) to a concentration of 1.25 mg/mL. 80 μL of the diluted samples were mixed with 10 μL of 0.5% RapiGest (from Waters, solved in 50 mM Tris(hydroxymethyl)aminomethane) and incubated for 5 minutes at 95° C. 4.5 μl of 0.02 M DTT (solved in 50 mM Tris(hydroxymethyl)-aminomethane) were added for reduction and incubated for 30 minutes at 37° C. For product digestion 5 μL of a 1 mg/mL Trypsin solution (solved in 50 mM acetic acid) were added and incubated for further 3 hours at 37° C. The reaction was stopped with 20 μl of 2% (v/v) trifluoroacetic acid and an incubation for 30 minutes at 37° C. The supernatant was diluted to 0.125 mg/mL with 50 mM Tris(hydroxymethyl)-aminomethane for analysis of the peptides.

UPLC Parameters:

The digested protein samples from the syringes were loaded onto an ACQUITY UPLC-CSH C-18 column from Waters, 100 mm×2.1 mm, 1.7 μm. 0.25 μg of the digested samples were eluted at 65° C. with a gradient of eluent A (water), eluent B (acetonitrile), eluent C (0.25% trifluoroacetic acid) and D (n-propanol) according to the following table.

TABLE 1
Elution profile used for RP-UPLC

| Time | Eluent A | Eluent B | Eluent C | Eluent D |
|---|---|---|---|---|
| [minutes] | [%] | [%] | [%] | [%] |
| 0.0 | 89.0 | 1.0 | 10.0 | 0.0 |
| 2.5 | 89.0 | 1.0 | 10.0 | 0.0 |
| 5.0 | 80.0 | 8.0 | 10.0 | 2.0 |
| 50.0 | 57.5 | 26.0 | 10.0 | 6.5 |
| 52.0 | 0.0 | 72.0 | 10.0 | 18.0 |
| 54.0 | 0.0 | 72.0 | 10.0 | 18.0 |
| 56.0 | 89.0 | 1.0 | 10.0 | 0.0 |
| 60.0 | 89.0 | 1.0 | 10.0 | 0.0 |

Method parameters for mass spectrometry:

| Ionisation type: | ESI | | Polarity: | Positive | |
|---|---|---|---|---|---|
| Analyser mode: | Sensitivity | | Experiment type: | MS | |
| Start Mass: | 50 | m/z | Cone Gas Flow: | 30 | L/h |
| End Mass: | 2000 | m/z | Desolvation Gas Flow: | 1000 | L/h |
| Source Temperature: | 120° | C. | Scan Time: | 0.5 | s |
| Desolvation | 450° | C. | Capillary Voltage: | 3.0 | kV |
| Temperature: | | | | | |
| Cone Voltage: | 35 | V | | | |

LockSpray Profile

Reference Compound: Leucine Enkephalin

MS Lock mass: 556.2766 m/z

Scan Time: 0.5 s

Interval: 30 s

1.4 Titer Quantification

Octet HTX (Pall ForteBio) was used for titer quantification. Octet HTX utilizes Bio-layer Interferometry (BLI), which is an optical analytical technique for measuring biomolecular interactions using specifically designed biosensors. Interactions are measured in real-time, with the ability to determine analyte concentrations. As standard the Eylea® reference product was used.

1.5 Cell Counting

Cell number and viability were determined with the Vi-CELL Cell Counter & Cell Viability Analyzer (Beckmann) which uses trypan blue dye exclusion.

2. Protein Production in Shake Flasks

All experiments were performed with a CHO cell line expressing aflibercept.

The basal cell culture medium used was EX-CELL® Advanced CHO fed batch medium (Sigma Aldrich). All experiments were performed with shake flasks (SF) of 250 mL scale (50 mL working volume).

For feeding the cells, the feed medium EX-CELL® Advanced CHO feed 1 (with glucose; Sigma Aldrich) supplemented with 25 mM N-acetyl-glucosamine (GlcNAc) (Sigma Aldrich) was added to the cell culture. The cell culture supplements manganese chloride tetrahydrate, glutamine and methionine (all from Sigma Aldrich) were added separately from the feed medium.

In total 7 SF experiments were performed, referred to as SF runs 1-7. Seeding density for the 250 mL shake flask experiments was 0.40×10⁶ cells/mL in 50 mL working volume. The shake flasks were run in fed-batch mode, with temperature, CO₂ and pH closely monitored. The feeding process was initiated three days after the inoculation. Cultures were fed with the medium as described above and additionally with manganese chloride tetrahydrate (to a final concentration of 0.2 mM), L-methionine and L-glutamine. The latter two were added to the cultures at varying concentrations, as described in Table 2.

In addition, all cultures were fed with 45% D-(+)-Glucose (Sigma Aldrich) solution once daily in case the concentration was below 4 g/L (analyzed by Nova Bioprofile 400, Nova Biomedical). If glucose was measured to be <4 g/L, D-(+)-Glucose was fed to 6 g/L.

The cell culture temperature was controlled at 37° C. from day 0 to day 6. In all SF experiments a T-shift to 34° C. on day 6 was applied (37° C.→34° C.). In addition, for some selected experiments another T-shift to 32° C. on day 10 was introduced (34° C.→32° C.) to assess its impact on product oxidation (see Table 2). The cultures were incubated for 14 days with shaking at 85 revolutions per minute (rpm; Multitron shaker incubator, Infors). The operating conditions of the SF experiments are summarized in Table 3.

TABLE 2
Feeding schedule for the SF experiments

| | EX- | | | | | | | | | |
|---|---|---|---|---|---|---|---|---|---|---|
| | CELL ® | | | | | | | | | |
| | Advanced | Manganese | | | | | | | | |
| | CHO Feed | chloride | | | | | | | | |
| | 1 with 25 | tetrahydrate | | | | | | | | |
| | mM | (Stock: 2 | L-Glutamine | | | | L-Methionine | | | |
| | GlcNAc | mM) | (Stock: 200 mM) | | | | (Stock: 50 mg/mL) | | D-(+)-Glucose 45% | T-shift (34 → 32° C.) |
| Day/SF | All shake flasks | | 1 | 2 | 4, 5, | 3 | 5, 6 | 7 | All shake flasks | 6, 7 |
| run | | | | | 6, 7 | | | | | |
| 0; | | | | | | | | | If glucose was measured | |
| Inocula- | | | | | | | | | to be <4 g/L, glucose was | |
| tion | | | | | | | | | fed to 6 g/L once daily | |
| 1 | | | | | | | | | | |
| 2 | | | | | | | | | | |
| 3 | 2.5 mL | 1.0 mL | | | | | | | | |
| 4 | | | | Up to 2 mM | | | | | | |
| 5 | 2.5 mL | 1.0 mL | | | | | | | | |
| 6 | T-shift (37° C. → 34° C.) | | | | | | | | | |
| 7 | 2.5 mL | 1.0 mL | | | Up to 0.5 | Up to | | | | |
| | | | | | mM | 1 mM | | | | |
| 8 | | | | | | | | | | |
| | 2.5 mL | 1.0 mL | | | | | 0.5 ml | 0.5 ml | | |
| 10 | | | | | Up to 0.5 | Up to | | 0.5 ml | | T-shift (34° C. |
| | | | | | mM | 1 mM | | | | →32° C.) |
| 11 | 2.5. mL | 1.0 mL | | | | | | | | |
| 12 | | | | | | | | | | |
| 13 | | | | | | | | | | |
| 14 | | | | | | | | | | |

TABLE 3
Process parameters for SF experiments
Incubator

| Parameter | Set Point |
|---|---|
| Inoculation VCD [×10⁶ viable | 0.4 |
| cells/mL] | |
| Temperature from day 0 to day 6 | 37 |
| [° C.] | |
| T-shift on day 6 [° C.] | 34 |
| T-shift on day 10* [° C.] | 32 |
| CO₂ [%] | 5 |
| Culture Duration [days] | 14 |
| Agitation [rpm] | 85 (for 50 mm |
| | orbit platforms) |

*for some selected shake flask experiments (see Table 2 and Table 4)

The condition of the cultures was evaluated by measuring the viable cell density (VCD) and cell viability from day 4 through day 14 (only day 14 data are shown, Table 4) with the Vi-CELL Cell Counter & Cell Viability Analyzer (Beckmann). In addition, product titer was measured on day 5 and 7 and from day 10 through day 14 (only day 14 data are shown, Table 4) by Octet HTX (Pall ForteBio). Shake flasks were harvested on day 14 of fed-batch cultivation. Harvests were clarified and subsequently purified via Protein A chromatography. The purified material was then analyzed for product oxidation by reduced peptide mapping. In addition, the purified material was subjected to SEC-HPLC analysis for quantifying the amount of monomer content [%] (percentage of peak area for the monomer in relation to the total peak area of the eluted species). The aim of SEC-HPLC analytics was to demonstrate that L-Glutamine and L-Methionine have no adverse impact on product stability. Details on the different methods used are provided above.

The results are depicted in Table 4. Of note, oxidation results are depicted only for Met192, as the other methionine residues in the aflibercept protein did not show any changes in oxidation upon feeding with L-Methionine, L-Glutamine and implementation of a day 10 temperature shift (34° C.→32° C.). For tryptophane residues no oxidation could be detected.

TABLE 4
Effect of L-Glutamine, L-Methionine and day 10 T-shift
(34° C. → 32° C.) on product oxidation, process
performance parameters and SEC monomer content at SF scale

| | SF run | | | | | | |
|---|---|---|---|---|---|---|---|
| | 1 | | | | | | |
| Culture | (control | SF run | SF run | SF run | SF run | SF run | SF run |
| conditions | run) | 2 | 3 | 4 | 5 | 6 | 7 |
| L- | — | √ | √ | √ | √ | √ | √ |
| Glutamine | | | | | | | |
| feeding on | | | | | | | |
| day 4 up to | | | | | | | |
| 2 mM | | | | | | | |
| L- | — | — | 0.5/0.5 | 1.0/1.0 | 0.5/0.5 | 0.5/0.5 | 0.5/0.5 |
| Glutamine | | | | | | | |
| feeding on | | | | | | | |
| day 7/10 up | | | | | | | |
| to x mM | | | | | | | |
| L- | — | — | — | — | 500/— | 500/— | 500/500 |
| Methionine | | | | | | | |
| feeding on | | | | | | | |
| day 9/10 | | | | | | | |
| [mg/L] | | | | | | | |
| T-shift on | — | — | — | — | — | √ | √ |
| day 10: | | | | | | | |
| 34° C. → | | | | | | | |
| 32° C. | | | | | | | |

Results process performance

| | | | | | | | |
|---|---|---|---|---|---|---|---|
| Titer day | 2.2 | 2.1 | 2.2 | 2.2 | 2.2 | 2.2 | 2.1 |
| 14 [mg/mL] | | | | | | | |
| Cell | 85 | 80 | 79 | 80 | 80 | 85 | 87 |
| viability | | | | | | | |
| day 14 [%] | | | | | | | |
| Max VCD | 15.3 | 14.6 | 14.2 | 15.1 | 16.8 | 14.9 | 15.6 |
| [*10\hat{ }6 | | | | | | | |
| cells/mL] | | | | | | | |

Results product quality

| | | | | | | | |
|---|---|---|---|---|---|---|---|
| Met192 | 19.0 | 13.0 | 11.4 | 11.5 | 10.1 | 8.1 | 7.8 |
| oxidation | | | | | | | |
| [%] | | | | | | | |
| (PepMap | | | | | | | |
| red) | | | | | | | |
| Monomer | 97 | 96 | 96 | 96 | 97 | 97 | 97 |
| content [%] | | | | | | | |
| (SEC- | | | | | | | |
| HPLC) | | | | | | | |

The results shown in Table 4 indicate that L-Glutamine had hardly any impact on cell growth characteristics or titer. However, surprisingly, L-Glutamine feeding on day 4 up to 2 mM significantly reduced Met192 oxidation (from 19.0 to 13.0%; see SF2 vs SF1). Further improvements in Met192 oxidation were achieved by L-Glutamine feeding on day 7 and 10 up to 0.5 mM (SF3 vs SF2), L-Methionine feeding on day 9 at 500 mg/L (SF5 vs SF3) and introduction of a temperature shift from 34° C. to 32° C. (SF6 vs SF5) on day 10 of fed-batch cultivation (first T-shift (37° C.→34° C.) occurred on day 6). Combining the aforementioned factors in one run (SF6) led to a significant decrease in Met192 oxidation from originally 19.0% to 8.1% (see SF6 vs SF1).

On the other hand, feeding of increased amounts of L-Glutamine (1.0 mM vs 0.5 mM on day 7 and 10; see SF4 vs SF3) and L-Methionine (500 mg/L on day 9 and 10 vs 500 mg/L on day 9; see SF7 vs SF6) had no or hardly any further beneficial impact on Met192 oxidation.

Furthermore, it was confirmed that L-Glutamine and L-Methionine did not have any major impact on cell growth characteristics, titer and SEC monomer content in the range of concentration tested (Table 4).

3. Protein Production in Bioreactors

2×5 L bioreactors (BR) (4 L working volume), referred to as BR run 1 and 2, were run with exactly the same conditions as applied for SF1 (control run) and SF6 shown in Tables 2 and 4, respectively. This was done to assess whether the improvement in Met192 oxidation observed at shake flask scale can be reproduced at bioreactor scale (results see Table 6).

The operating conditions of the BR runs are summarized in Table 5.

TABLE 5
Process parameters for BR runs

| | |
|---|---|
| Parameter | Set Point |

Bioreactor

| | |
|---|---|
| Inoculation VCD [×10⁶ | 0.4 |
| viable cells/mL] | |
| Temperature from day 0 | 37 |
| to day 6 [° C.] | |
| T-shift on day 6 [° C.] | 34 |
| T-shift on day 10* [° C.] | 32 |
| pH | 7.1 +/− 0.3 |
| dO₂ [%] | 30 |
| Culture Duration [Days] | 14 |
| Agitation [rpm] | 210 (dual 30° impeller set-up) |

Acid, Base, and Antifoam addition

| | |
|---|---|
| Acid | CO₂ on demand through a ring sparger |
| Base | 2M Sodium hydroxide (Merck Millipore) on |
| | demand by pH control, through base pump |
| Antifoam | Antifoam FoamAway (Thermo Fisher |
| | Scientific). |
| | If the foam level was >2 cm, 1 mL of antifoam |
| | was added. If required, additional 1 mL |
| | amounts of antifoam was added until the foam |
| | level was <2 cm. |

Control Strategy

| | |
|---|---|
| DO Control | DO was controlled at 30% using optical probes. |
| | A constant air flow of 200 mL/min in the |
| | headspace and O₂ on demand through a ring |
| | sparger was used. |
| pH Control | Set-point 7.1 with a dead band of ±0.3. |

*applies to BR run 2 only

TABLE 6
Effect of L-Glutamine, L-Methionine and day 10 temperature
shift (34° C. → 32° C.) on Met192 oxidation,
process performance and SEC monomer content at 5 L BR scale

| Culture conditions | BR run 1 | BR run 2 |
|---|---|---|
| L-Glutamine feeding on | — | ✓ |
| day 4 up to 2 mM | | |
| L-Glutamine feeding on | — | 0.5/0.5 |
| day 7/10 up to x mM | | |
| L-Methionine feeding on | — | 500/— |
| day 9/10 [mg/L] | | |
| T-shift on day 10: 34° C. | — | ✓ |
| → 32° C. | | |

Results process performance

| | | |
|---|---|---|
| Titer day 14 [mg/mL] | 2.1 | 2.1 |
| Cell viability day 14 [%] | 82 | 83 |
| Max VCD [*10\hat{ }6 | 18.3 | 17.9 |
| cells/mL] | | |

Results product quality

| | | |
|---|---|---|
| Met192 oxidation [%] | 18.2 | 7.5 |
| (PepMap red) | | |
| Monomer content [%] | 95 | 96 |
| (SEC-HPLC) | | |

As can be seen from Table 6, the conditions used in bioreactor run 2 led to a significant decrease of Met192 oxidation (from 18.2 to 7.5%), very similar to what was seen during the shake flask experiments (Table 4, SF6 vs SF1). Thus, it could be demonstrated that feeding of L-Glutamine and L-Methionine combined with a T-shift on day 10 of fed-batch cultivation (34° C.→32° C.) resulted in a strongly improved oxidation resistance of Met192 both at SF (see Table 4, SF6 vs SF1) and bioreactor scale (Table 6, BR2 vs BR1). Furthermore, the optimized culture conditions did not have any major impact on cell growth characteristics, titer and SEC monomer content.

Some embodiments of the present invention relate to:
1. Method for reducing the percentage of oxidized methionine residues in a recombinant protein, said method comprising culturing cells expressing said recombinant protein in a suitable cell culture medium and feeding the cells with glutamine.
2. Method according to item 1, comprising feeding the cells with 0.2 mM to 5 mM glutamine.
3. Method according to item 1 or 2, comprising feeding the cells with 0.5 mM to 4 mM glutamine on day 3 to 5 of the culture.
4. Method according to any one of the preceding items, comprising feeding the cells with 0.2 mM to 2 mM glutamine on day 6 to 12 of the culture.
5. Method according to any one of the preceding items, further comprising feeding the cells with methionine.
6. Method according to item 5, comprising feeding the cells with 250 to 1000 mg/l methionine.
7. Method according to item 5 or 6, comprising feeding the cells with methionine on day 8 to 12 of the culture.
8. Method according to any one of the preceding items, further comprising reducing the culture temperature.
9. Method according to item 8, wherein the culture temperature is reduced from 37° C. to a temperature within the range of 30° C. to 35° C.
10. Method according to item 8 or 9, wherein the culture temperature is reduced in a first step from a first temperature of 37° C. to a second temperature within the range of 33° C. to 35° C. and in a second step from the second temperature of 33° C. to 35° C. to a third temperature within the range of 31° C. to 33° C.
11. Method according to item 10, wherein the first step is performed on day 5 to 7 of the culture.
12. Method according to item 10 or 11, wherein the second step is performed on day 9 to 12 of the culture.
13. Method according to any one of the preceding items, wherein the recombinant protein is a recombinant antibody or a fusion protein of a receptor and the Fc portion of an immunoglobulin.
14. Method according to any one of the preceding items, wherein the recombinant protein is aflibercept.
15. Method according to item 14, wherein the methionine is methionine 192 of aflibercept.
16. Method according to any one of the preceding items, wherein the cells are mammalian cells.
17. Method according to any one of the preceding items, wherein the cells are CHO cells.
18. Use of glutamine for reducing the percentage of oxidized methionine residues in a composition comprising a recombinant protein.
19. Method for producing a recombinant protein, comprising
(a) culturing cells expressing the recombinant protein in a suitable cell culture medium;
(b) feeding the cells with glutamine, wherein the cells are fed with 0.5 mM to 4 mM glutamine on day 3 to 5 of the culture and with 0.2 mM to 2 mM glutamine on day 6 to 12 of the culture; and
(c) obtaining the recombinant protein.
   20. Method according to item 19, wherein the recombinant protein has a reduced percentage of oxidized methionine residues compared to a protein produced without feeding the cells with glutamine.
   21. Method according to any one of items 19 and 20, further comprising feeding the cells with methionine.
   22. Method according to item 21, comprising feeding the cells with 250 to 1000 mg/l methionine.
   23. Method according to item 21 or 22, comprising feeding the cells with methionine on day 8 to 12 of the culture.
   24. Method according to any one of items 19 to 23, further comprising reducing the culture temperature.
   25. Method according to item 24, wherein the culture temperature is reduced from 37° C. to a temperature within the range of 30° C. to 35° C.
   26. Method according to item 24 or 25, wherein the temperature is reduced in a first step from a first temperature of 37° C. to a second temperature within the range of 33° C. to 35° C. and in a second step from the second temperature of 33° C. to 35° C. to a third temperature within the range of 31° C. to 33° C.
   27. Method according to item 26, wherein the first step is performed on day 5 to 7 of the culture.
   28. Method according to item 26 or 27, wherein the second step is performed on day 9 to 12 of the culture.
   29. Method according to any one of items 19 to 28, wherein the recombinant protein is a recombinant antibody or a fusion protein of a receptor and the Fc portion of an immunoglobulin.
   30. Method according to any one of items 19 to 29, wherein the recombinant protein is aflibercept.
   31. Method according to item 30, wherein the methionine is methionine 192 of aflibercept.
   32. Method according to any one items 19 to 31, wherein the cells are mammalian cells.
   33. Method according to any one of items 19 to 32, wherein the cells are CHO cells.
   34. Method for producing aflibercept, comprising:
(a) culturing cells expressing aflibercept in a suitable cell culture medium;
(b) feeding the cells with glutamine; and
(c) obtaining the aflibercept.
   35. Method according to item 34, comprising feeding the cells with 0.2 mM to 5 mM glutamine.
   36. Method according to item 34 or 35, comprising feeding the cells with 0.5 mM to 4 mM glutamine on day 3 to 5 of the culture.
   37. Method according to any one of items 34 to 36, comprising feeding the cells with 0.2 mM to 2 mM glutamine on day 6 to 12 of the culture.
   38. Method according to any one of items 34 to 37, further comprising feeding the cells with methionine.
   39. Method according to item 38, comprising feeding the cells with 250 to 1000 mg/l methionine.
   40. Method according to item 38 or 39, comprising feeding the cells with methionine on day 8 to 12 of the culture.
   41. Method according to any one of items 34 to 40, further comprising reducing the culture temperature.
   42. Method according to item 41, wherein the culture temperature is reduced from 37° C. to a temperature within the range of 30° C. to 35° C.
   43. Method according to item 41 or 42, wherein the temperature is reduced in a first step from a first temperature of 37° C. to a second temperature within the range of 33° C. to 35° C. and in a second step from the second temperature of 33° C. to 35° C. to a third temperature within the range of 31° C. to 33° C.
   44. Method according to item 43, wherein the first step is performed on day 5 to 7 of the culture.
   45. Method according to item 43 or 44, wherein the second step is performed on day 9 to 12 of the culture.
   46. Method according to any one of items 34 to 45, wherein the methionine is methionine 192 of aflibercept.
   47. Method according to any one of items 34 to 46, wherein the cells are mammalian cells.
   48. Method according to any one of items 34 to 47, wherein the cells are CHO cells.

1. A method for reducing the percentage of oxidized methionine residues in a recombinant protein, said method comprising culturing cells expressing said recombinant protein in a suitable cell culture medium and feeding the cells with glutamine.

2. The method according to claim 1, comprising feeding the cells with 0.2 mM to 5 mM glutamine.

3. The method according to claim 1, comprising feeding the cells with 0.5 mM to 4 mM glutamine on day 3 to 5 of the culture.

4. The method according to claim 1, comprising feeding the cells with 0.2 mM to 2 mM glutamine on day 6 to 12 of the culture.

5. The method according to claim 1, further comprising feeding the cells with methionine.

6. The method according to claim 5, comprising feeding the cells with 2:50 to 1000 mg/l methionine.

7. The method according to claim 1, further comprising reducing the culture temperature.

8. The method according to claim 7, wherein the culture temperature is reduced from 37° C. to a temperature within the range of 30° C. to 35° C.

9. The method according to claim 7, wherein the temperature is reduced in a first step from a first temperature of 37° C. to a second temperature within the range of 33° C. to 35° C. and in a second step from the second temperature of 33° C. to 35° C. to a third temperature within the range of 31° C. to 33° C.

10. The method according to claim 1, wherein the recombinant protein is a recombinant antibody or a fusion protein of a receptor and the Fc portion of an immunoglobulin.

11. The method according to claim 1, wherein the recombinant protein is aflibercept.

12. The method according to claim 1, wherein the cells are CHO cells.

13. (canceled)

14. A method for producing a recombinant protein, comprising
(a) culturing cells expressing the recombinant protein in a suitable cell culture medium;
(b) feeding the cells with glutamine, wherein the cells are fed with 0.5 mM to 4 mM glutamine on day 3 to 5 of the culture and with 0.2 mM to 2 mM glutamine on day 6 to 12 of the culture; and
(c) obtaining the recombinant protein.

15. A method for producing aflibercept, comprising:
(a) culturing cells expressing aflibercept in a suitable cell culture medium;
(b) feeding the cells with glutamine; and
(c) obtaining the aflibercept.
